(19) United States
(12) Patent Application Publication
(10) Pub. No.: US 2023/0196075 A1
(43) Pub. Date: Jun. 22, 2023

(54) INFERRING DEVICE, TRAINING DEVICE AND INFERRING METHOD

(71) Applicant: Preferred Networks, Inc., Chiyoda-ku, Tokyo-to (JP)

(72) Inventor: Ryuichiro ISHITANI, Tokyo-to (JP)

(73) Assignee: Preferred Networks, Inc., Chiyoda-ku, Tokyo-to (JP)

(21) Appl. No.: 18/167,948

(22) Filed: Feb. 13, 2023

Related U.S. Application Data
(63) Continuation of application No. PCT/JP2021/029717, filed on Aug. 12, 2021.

(30) Foreign Application Priority Data
Aug. 14, 2020 (JP) 2020-137071

(51) Int. Cl. G06N 3/0455; G06N 3/0985

(52) U.S. Cl. CPC G06N 3/0455; G06N 3/0985

(57) ABSTRACT

An inferring device includes one or more memories and one or more processors. The one or more processors are configured to generate information on a tree including information on a node and information on an edge from a latent representation by using a trained inference model; and generate a graph from the information on the tree. The information on the tree includes connection information on the nodes.

### CROSS REFERENCE TO RELATED APPLICATION

This application is continuation application of International Application No. JP2021/029717, filed on Aug. 12, 2021, which claims priority to Japanese Patent Application No. 2020-137071, filed on Aug. 14, 2020, the entire contents of which are incorporated herein by reference.

### FIELD

The present disclosure relates to an inferring device, a training device, and an inferring method.

### BACKGROUND

Methods of generating a molecular structure using a learned generative model are studied. These methods each execute training of the generative model using a molecular structure as teacher data or the like to make the generative model learn mapping between the molecular structure and a latent vector with a fixed length corresponding to the molecular structure. At the time of generating a molecular structure, the generative model is caused to randomly change a latent vector or search for a specific property as a target and infer a molecular structure corresponding to the latent vector.

In the generative model of the molecular structure, a representation method of the molecular structure (for example, in a character string format or a graph format) greatly influences the performance of the generation of the molecular structure. For example, when representing it in the character string format, there may occur a case where a character string representation output from the generative model does not correspond to an effective molecular structure, thus affecting the performance. When representing it in the graph format, there is no case where a graph representation does not correspond to an effective molecular structure, but it is difficult to cause the generative model to efficiently learn a molecular structure (for example, a phenyl group or the like) frequently appearing in an organic compound.

In contrast to the above, there are proposed methods of coarse-graining the graph representation of the molecular structure (for example, a tree representation of representing a benzene ring as one node or the like) and learning and generating the tree representation. As one of them, there is a proposed method using a tree representation obtained by decomposing the graph representation of the molecular structure into a tree. However, these methods have such a disadvantage that it is impossible to uniquely return the coarse-grained tree representation to the molecular structure. For this reason, reversible conversion to the molecular structure is realized by using not only the tree representation but also the graph representation in combination. However, the combination use causes such a problem that the amount of calculation is large and it takes a long time for learning and generation.

### BRIEF DESCRIPTION OF DRAWINGS

FIG. 1 is a block diagram illustrating a configuration of an inferring device according to an embodiment;

FIG. 2 is a diagram illustrating conversion in inference according to an embodiment;

FIG. 3 is a block diagram illustrating a configuration of a training device according to an embodiment;

FIG. 4 is a diagram illustrating conversion in training according to an embodiment;

FIGS. 5-8 are diagrams illustrating examples of site information according to an embodiment;

FIG. 9 is a diagram illustrating an example of assemble according to an embodiment; and

FIG. 10 is a block diagram illustrating a configuration of an inferring device or a training device according to an embodiment.

### DETAILED DESCRIPTION

According to one embodiment, an inferring device includes one or more memories and one or more processors. The one or more processors are configured to generate information on a tree including information on a node and information on an edge from a latent representation by using a trained inference model; and generate a graph from the information on the tree. The information on the tree includes connection information on the nodes.

First, terms in this disclosure will be explained.

“Tree decomposition” means a method of mapping a graph representation of a molecular structure into a tree representation.

“Singleton” means a node corresponding to an atom being a branch point in the case of decomposing the graph representation of a compound molecule into a tree. The branch point of a graph is a node of the singleton when the branch point does not belong to a ring structure.

“Bond” expresses two atoms which are covalently bonded as one node. However, in the case of a covalent bond belonging to a ring structure, the following “ring” is applied.

“Ring” is a node corresponding to the ring structure in the case of decomposing the graph representation of a compound molecule into a tree. Representative examples of the compound expressed as the ring include benzene, pyridine, and pyrimidine, or cyclobutadiene, cyclopentadiene, pyrrole, and cyclooctatetraene, cyclooctane, but the compound is not limited to them and only needs to be a cyclic one.

“Site information” is information indicating a relationship of how the tree-decomposed nodes are connected in the original molecular structure. General tree decomposition is irreversible, but the use of this site information ensures reversibility capable of reverse conversion from the tree representation obtained by the tree decomposition to the graph representation in an embodiment of this disclosure. Further, the tree representation added with the site information is called a tree representation with site information.

In this disclosure, “node” and “edge” mainly represent a node and an edge in the tree representation and, before and after the tree decomposition, the node and the edge in the graph representation and the node and the edge in the tree representation will be replaced as appropriate.

Hereinafter, embodiments of the present invention will be explained with reference to the drawings. The explanations of the drawings and the embodiments are presented by way of example only, and are not intended to limit the present invention. In this disclosure, data represented in a graph may be described as a first type of data, data represented in a tree with site information may be described as a second type of data, and a latent representation may be described as a third type of data.

### Inferring Device

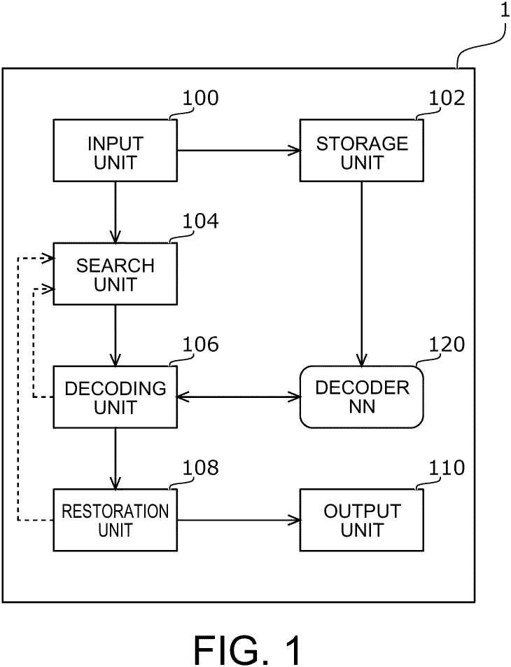

FIG. 1 is a block diagram illustrating an example of a configuration of an inferring device according to an embodiment. An inferring device 1 includes an input unit 100, a storage unit 102, a search unit 104, a decoding unit 106, a restoration unit 108, and an output unit 110. The inferring device 1 generates and outputs a chemical formula of a compound based on information input from the input unit 100 or on a latent state automatically generated in the inferring device 1.

The input unit 100 accepts input from an external part. Data received by the input unit 100 is stored in the storage unit 102 when necessary. For example, the input unit 100 receives a request from a user, and accepts information being a seed for search in the search unit 104.

The storage unit 102 stores information required for the operation of the inferring device 1. The storage unit 102 may further store intermediate generated data, final generated data, and so on in processing by the inferring device 1. For example, when information processing by software is concretely realized using hardware resources in various operations of the inferring device 1, the storage unit 102 stores a program relating to the software. The storage unit 102 may further store hyperparameters and parameters constituting a trained neural network model.

The search unit 104 acquires data in the latent representation (third type) for generating a compound. For example, its latent variable may be acquired using a random number value. As another example, the search unit 104 may acquire, as the latent variable, a value obtained by addition or multiplication of a random number value and an input latent variable or an already acquire latent variable based on the compound.

As still another example, the search unit 104 may optimize the latent representation so as to acquire a better result based on a result decoded by the decoding unit 106 or on a result restored by the restoration unit 108. For this optimization, for example, a method based on meta-heuristics such as PSO (Particle Swarm Optimization) or the like may be used.

The decoding unit 106 decodes the latent variable searched for by the search unit 104 using the trained neural network model. The decoding unit 106 decodes the latent variable and thereby acquires the tree representation with site information corresponding to the latent variable.

The decoding unit 106 constitutes a decoder neural network (hereinafter, described as a decoder NN 120) using, for example, the various parameters stored in the storage unit 102. The decoding unit 106 then inputs the latent variable into the decoder NN 120 and thereby acquires data in the tree representation with site information (second type). The decoder NN 120 is a model optimized, for example, by a method of generating an autoencoder.

More specifically, the decoding unit 106 converts data from the third type to the second type using the decoder NN 120 which outputs the second type of data when the third type of data is input thereinto.

For example, this neural network model is made by combining an encoder which outputs a latent variable when a tree representation with site information is input thereinto and a decoder which outputs a tree representation with site information when a latent variable is input thereinto, and optimizing them. For example, the dimension of the latent variable being the input to the decoder NN 120 may be a dimension lower than the dimension of information of the tree representation with site information being the output.

The restoration unit 108 outputs a chemical structural formula of a compound from the tree representation with site information output from the decoding unit 106. The restoration unit 108 executes a reverse operation of the tree decomposition and thereby converts the tree representation with site information to data in a chemical structural formula representation (graph representation, first type). In other words, the restoration unit 108 converts data from the second type to the first type. The reverse conversion to the graph representation is called assemble. A method of assemble will be explained in more detail in the later explanation of a training device.

Note that at least one of the decoding unit 106 and the restoration unit 108 may perform an operation of determining an acquired result. For example, the decoding unit 106 may evaluate the output tree representation with site information by an evaluation function, and select whether to output or re-search based on its score value. Similarly, the restoration unit 108 may evaluate information on the output chemical structural formula by an evaluation function, and select whether to output or re-search based on its score value.

The search unit 104 executes re-search when at least one of the decoding unit 106 and the restoration unit 108 determines to perform re-search. Note that this determination is not executed in the decoding unit 106 or the restoration unit 108, but may be made by the search unit 104 based on the output from the decoding unit 106 or the restoration unit 108.

The search unit 104 may search for a plurality of latent variables at the same timing. For example, in the case of using PSO as a search method, the search unit 104 executes a search for the latent variables being a plurality of particles in parallel. In this case, the decoding unit 106 may also generate a plurality of pieces of information on a tree from the plurality of latent variables in parallel. Furthermore, the restoration unit 108 may also generate a plurality of pieces of information on the chemical structural formula from the plurality of pieces of information on the tree in parallel. Here, the information on the tree represents the information on the tree including the information on the node and the information on the edge. Hereinafter, information including the information on the node and the information on the edge is simply described as the information on the tree.

The output unit 110 outputs appropriate information. The information output from the output unit 110 is, for example, a chemical structural formula. The output unit 110 outputs, for example, the information on the chemical structural formula to the external part. Alternatively, the output unit 110 may store the information on the chemical structural formula in the storage unit 102 and thereby output it. As explained above, the output is a concept including storage into the storage unit 102 as an internal part as well as output to the external part.

In the case where the search is completed in one session, the output unit 110 outputs that information. In the case where the output information is optimized by the search unit 104, the output unit 110 may output only final data or may output data at a plurality of steps including interim progress data together with the final data.

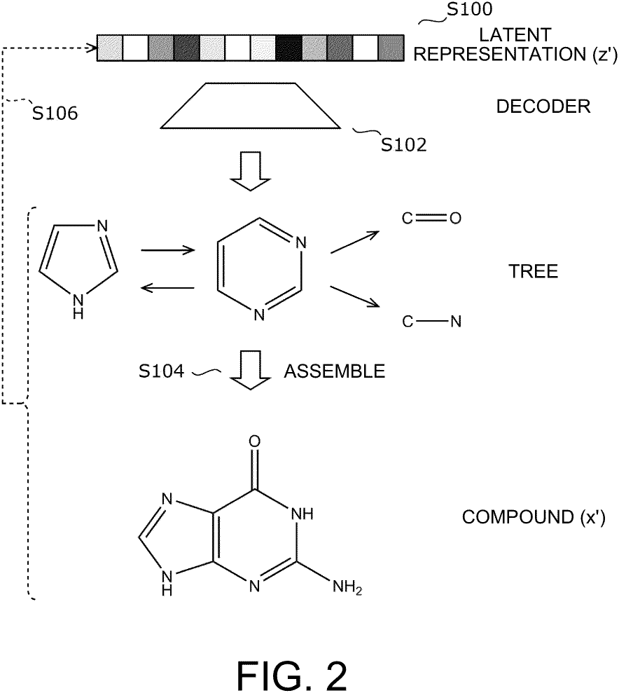

FIG. 2 illustrates the concept of inferring processing by the inferring device 1 as a chart. The flow of the processing by the inferring device 1 will be explained using FIG. 2.

First, the search unit 104 searches for a latent variable (third type of data) z′ (S100). As explained above, the latent representation may be acquired as a random number, or a latent variable close to a previously generated latent representation of a compound may be acquired. For example, when it is desired to infer a compound close in property or in structure to a certain compound, the structural formula (graph representation) of the compound is acquired as a latent representation z via the tree decomposition and the encoder. Then, by adding a minute value or the like to the latent representation z or multiplying the latent representation z by a value close to 1, the latent representation z′ being a search target is acquired.

Next, the decoding unit 106 inputs the latent representation z′ generated by the search unit 104 into the decoder NN 120 to acquire the data in the tree representation with site information (second type) (S102). An individual molecular formula illustrated in the drawing is a node in the tree representation with site information, and an arrow indicates the site information. The site information between the individual nodes is indicated as the arrow.

Next, the restoration unit 108 performs assemble using the tree representation with site information generated by the decoding unit 106 to acquire a graph representation x′ (first type of data) of the compound (S104).

If more search is necessary, the search unit 104 may acquire a new latent representation (third type of data) using the data generated by at least one of the decoding unit 106 and the restoration unit 108, and repeat the search (S106).

Then, finally acquired data on the compound is output, and then processing is completed. As explained above, the inferring device 1 can generate the information on the compound x′.

### Training Device

Next, a configuration and an operation of the training device will be explained.

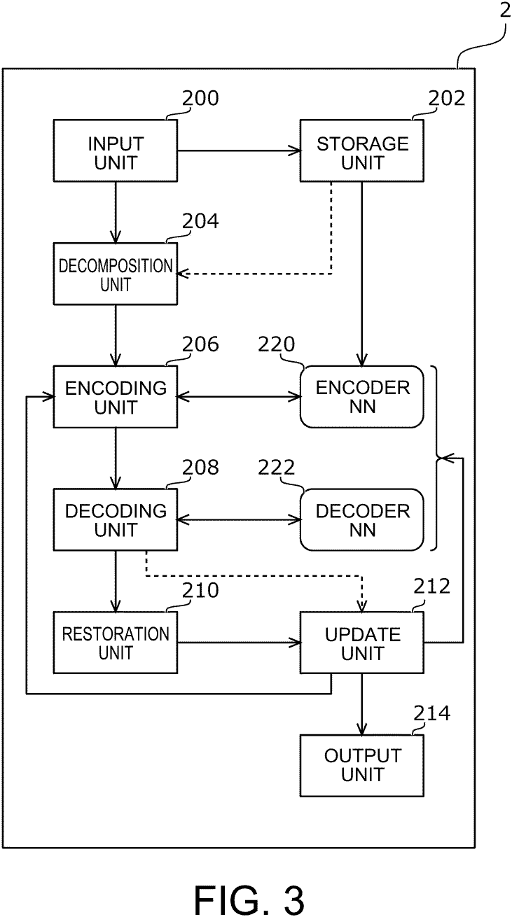

FIG. 3 is a block diagram illustrating a configuration of a training device according to an embodiment. A training device 2 includes an input unit 200, a storage unit 202, a decomposition unit 204, an encoding unit 206, a decoding unit 208, a restoration unit 210, an update unit 212, and an output unit 214.

The input unit 200 accepts input of data required for the operation of the training device 2. The training device 2 acquires, for example, data in the graph representation (first type) of a compound being training data via the input unit 200. The input unit 200 may further accept input of hyperparameters and the like of a neural network model for optimization.

The storage unit 202 stores data required for the operation of the training device 2. The storage unit 202 may store the training data acquired by the training device 2 via the input unit 200. The other operations are substantially the same as those of the storage unit 102 of the inferring device 1, and therefore detailed explanation thereof will be omitted.

The decomposition unit 204 executes the tree decomposition on the first data acquired via the input unit 200 and thereby converts the data to data in the tree representation with site information (second type). As another example, the decomposition unit 204 may acquire the second type of data based on the first type of data stored in the storage unit 202. In other words, the decomposition unit 204 converts data from the first type to the second type.

The encoding unit 206 inputs the second data generated by the decomposition unit 204 into an encoder NN 220 to acquire data in the latent representation (third type). The encoding unit 206 forms the encoder NN 220, for example, based on various parameters of the model stored in the storage unit 202 to generate the third type of data from the second type of data. In other words, the encoding unit 206 converts data from the second type to the third type.

The decoding unit 208 inputs the third type of data generated by the encoding unit 206 into a decoder NN 222 to generate the second type of data. The decoder NN 222 and the decoding unit 208 correspond to the above decoder NN 120 and decoding unit 106 of the inferring device 1, respectively. In short, the decoding unit 208 converts data from the third type to the second type.

The restoration unit 210 executes assemble on the second type of data output from the decoding unit 208 and thereby acquires the first type of data. The restoration unit 210 corresponds to the above restoration unit 108 of the inferring device 1. In short, the restoration unit 210 converts data from the second type to the first type.

The update unit 212 optimizes the encoder NN 220 and the decoder NN 222 based on the first type of data on the compound output from the restoration unit 210. For the optimization, various kinds of optimization used for optimization of the autoencoder may be used. Further, for the optimization of the autoencoder, a method of VAE (Vairational Autoencoder) handling a latent variable as a probability distribution may be used.

As another example, the update unit 212 does update the model based on the first type of data, but may update the model based on the second type of data output from the decoding unit 208. In this case, the optimization is executed by comparing the data obtained by converting the first type of data of the training data to the second type with the data output from the decoding unit 208.

The update unit 212 updates parameters of the encoder and the decoder using at least one of the first type of data and the second type of data as explained above. Desirably, the update unit 212 updates the parameters using the second type of data.

The output unit 214 outputs various parameters and the like of the encoder and decoder optimized by updating the parameters by the update unit 212. Similarly to the inferring device 1, the output unit 214 may output the acquired data to the external part or to the storage unit 202.

In the case where the parameters and the like are stored in the storage unit 202, the training device 2 after training may be used as the inferring device 1.

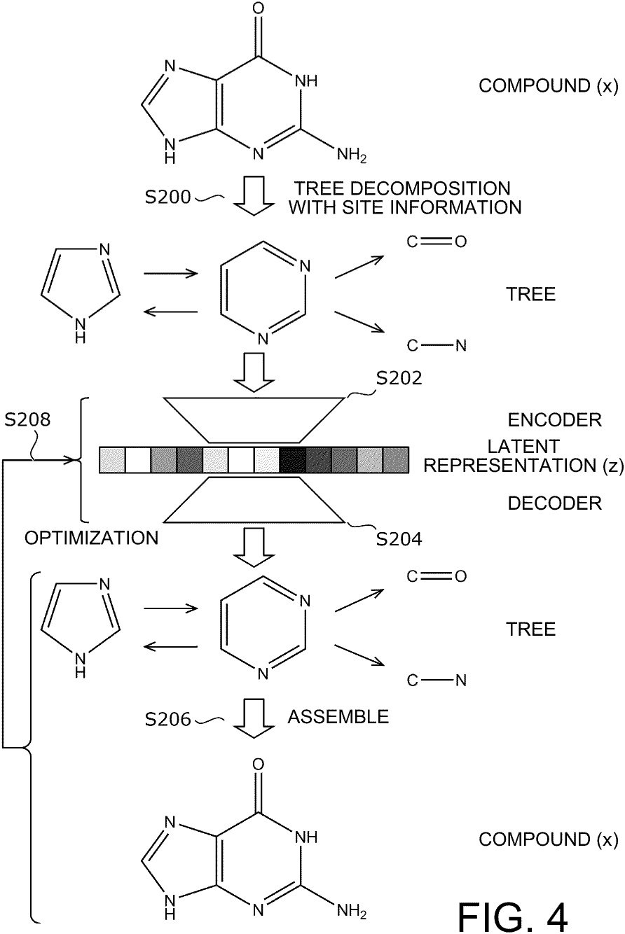

FIG. 4 illustrates the concept indicting training processing of the training device 2 as a chart. The flow of the processing of the training device 2 will be explained using FIG. 4.

First, the training device 2 acquires data x on a compound via the input unit 200. This data may be the first type of data, namely, data in a graphical-form representation.

Next, the decomposition unit 204 decomposes the first type of compound data into a tree to acquire the second type of data, namely, data in the tree representation with site information (S200).

Next, the encoding unit 206 inputs the second type of data generated by the decomposition unit 204 into the encoder NN 220 to generate a latent representation z being the third type of data (S202).

Next, the decoding unit 208 inputs the latent representation z being the third type of data generated by the encoding unit 206 into the decoder NN 222 to acquire the second type of data (S204).

Next, the restoration unit 210 executes assemble to acquire a graphical representation of the compound being the first type of data from the second type of data generated by the decoding unit 208 (S206).

Next, the update unit 212 updates the parameters of the encoder NN 220 and the decoder NN 222 based on the data restored by the restoration unit 210 (S208). Note that as explained above, the update unit 212 may update the parameters based not on the first type of data restored by the restoration unit 210 but on the second type of data generated by the decoding unit 208. In this case, the processing at S206 is not required.

Then, the finally acquired parameters and the like of the decoder NN 222 are output, and the processing is ended. As explained above, the training device 2 can generate at least the information on the decoder NN 222.

Note that the output unit 214 may output parameters and the like not only of the decoder NN 222 but also of the encoder NN 220. In this case, the parameters of the encoder NN 220 can be used for learning and the like in the future.

The decoder NN 222 optimized by the training device 2 can be used as the decoder NN 120 in the inferring device 1. Through use of the trained inference model, the inferring device 1 can realize the inference to generate the first type of data by designating one point in a third type of data space.

### Tree Decomposition

Next, tree decomposition with site information in this disclosure will be explained. The decomposition unit 204 and the restoration unit 210 of the training device 2 and the restoration unit 108 of the inferring device 1 execute conversion and assemble by the tree decomposition as in the following explanation.

The tree-decomposed node becomes a node of one of the above-explained singleton, bond, and ring. Examples of the connection between the nodes include four kinds of cases such as bond-bond, bond-singleton (or singleton-bond), ring-bond (or bond-ring), and ring-ring.

The bond-bond is a case where two bonds are connected to each other.

The bond-singleton is a case where one bond is connected to a singleton being a branch point.

The ring-bond is a case where one bond is connected to a ring.

The ring-ring is a case where two rings are directly connected. In this case, there are a condensing (connecting by sharing one bond) case and a spiro bonding (connecting by sharing only one atom) case.

When general tree decomposition is executed on the graph representation of a compound, information on a connection position or the like regarding cases related to a ring is lost. Therefore, restoration with uniqueness is impossible with only the tree information. In this embodiment, to uniquely convert between the tree information and the graph representation, connection information on nodes representing the relationship between the nodes connected to each other is given as information about a site to the tree information. This connection information includes, for example, information on the position where the nodes are connected and information on a direction in which the nodes are connected as will be explained in more detail below.

What kind of processing is performed in the tree decomposition and assemble will be explained in more detail for the above four cases.

The bond-bond connection will be explained. For example, in the case where node C—N and node C—N are connected by one edge, there may be a case where the bond nodes are connected by sharing carbon atoms and a case where the bond nodes are connected by sharing nitrogen atoms, in the original graphical representation. In other words, information on which of atoms have been shared in the original compound is lost in the tree representation, so that the graph representation cannot be restored from only the tree representation.

To cope with the above, the decomposition unit 204 stores in advance which atoms are shared between the nodes as the site information. Through use of the site information, the restoration unit 108, 210 can restore the graph representation.

In the case of the bond-singleton connection, the restoration is possible with only the tree representation by the general tree decomposition. For example, in the case where node C-N and node C are connected by one edge, it can be uniquely decided that both nodes share carbon atoms. Therefore, the restoration unit 108, 210 can acquire the graph representation without additional information.

In the case of the ring-bond connection, information on which atom in a ring structure of the ring node the bond node is connected to is lost in the general tree decomposition. Therefore, it is impossible to restore the graph representation from only the tree representation.

To cope with the above, the decomposition unit 204 stores in advance which position of the ring, namely, which atom the bond is connected to as the site information. The restoration unit 108, 210 can uniquely decide the connection position of the bond with respect to the ring based on the site information.

In the case of the ring-ring connection, the situation differs between the condensing case and the spiro bonding case.

In the condensing case, information on which bonds in ring structures two ring nodes have shared for condensation is lost by tree decomposition. Therefore, it is impossible to restore the graph representation from only the tree representation. To cope with the above, the decomposition unit 204 stores in advance which bonds have been shared as the site information. Further, the decomposition unit 204 stores in advance a direction in which the bonds are connected as site direction information. Through use of the site information and the site direction information, the restoration unit 108, 210 can restore the graph representation.

In the case of the spiro bonding, information on which atoms in ring structures two ring nodes have shared is lost by tree decomposition. Therefore, it is impossible to restore the graph representation from only the tree representation. To cope with the above, it is only necessary, for restoration, to store in advance which atoms have been shared as the site information as in the ring-bond connection.

The addition of the site information to the tree representation regarding the connection related with the ring enables restoration to the graph representation. Examples of the site information will be explained below.

The site information is appropriately decided in advance so as to uniquely represent, in a node, a partial atomic group of a compound represented by the node (two atoms connected by covalent bonding in the case of the bond node, three or more atoms belonging to a ring structure in the case of the ring node). In the case of the bond node, for example, a numerical value such as 0, 1 is given as the site information to each atom included in the node. In the case of the ring node, for example, numbers are given in a manner to pass through all atoms clockwise starting from an atom as a reference. The atom as the reference where the site information is 0 may be decided, for example, in ascending order when expressing element names by ASCII codes and sorting them by dictionary. As another example, the atom as the reference may be decided based on the atomic number.

In the bond node having a plurality of candidates for 0, any atom may be regarded as 0. In the case of the ring node, for example, an atom may be regarded as 0 in such an order that its dictionary order by ASCII code becomes the youngest when atoms are arranged in numerical order starting from the candidate, or its atomic number becomes smallest when atomic numbers are arranged in order. In the case of the atomic number, even if the number is 1 or 2 digits, it may be expanded to three digits for use. For example, in the case of the ring node having six atoms, the site information may be given so as to become the youngest as an 18-digit number.

Not limited to the above, but a method in which the same site information is appropriately given to the bond nodes or the ring nodes having the same configuration may be used. In the case of giving the site information by the same method, any atom may be regarded as a reference from among the atoms as candidates in the node with symmetry, such as a benzene ring, where there is no difference in the dictionary order.

The site information (including the site direction information) may be added to all of the nodes of the tree in implementation. For example, in the bond-singleton connection, the site information may be set to an arbitrary value, or may be set as 0. In the following explanation, it is assumed that the site information is added to all of the nodes. As a matter of course, in the bond-singleton connection, a configuration that the site information is not added is acceptable.

First, the site information to be added will be explained.

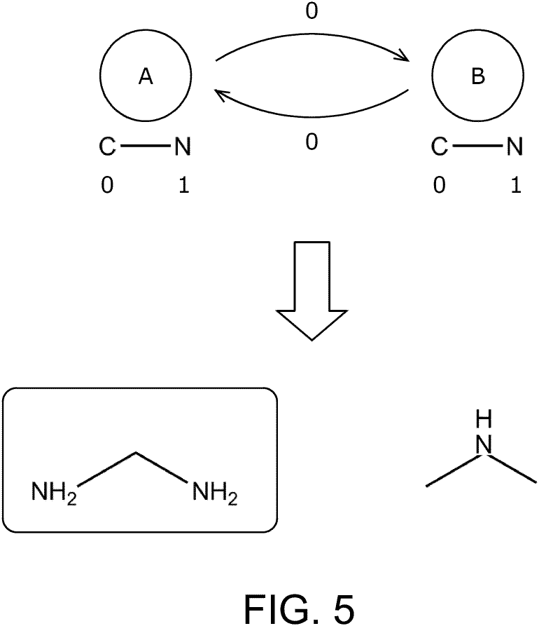

FIG. 5 is a diagram illustrating an example of the site information. FIG. 5 illustrates the site information regarding the bond-bond connection, and the bond-singleton case can be similarly processed. In the following diagrams, O indicates a node, and an arrow indicates a directed edge to which a feature is added.

For example, both of node A and node B are nodes indicating C—N, and these nodes are connected. It is assumed that a carbon atom (C) is numbered 0 and a nitrogen atom (N) is numbered 1 in the node. There may be two patterns of the molecular structure presented by this tree representation such as CH₃NHCH₃ and NH₂CH₂NH₂ as in FIG. 5. When information of 0 is added to edge A → B and information of 0 is added to edge B → A, a graph representation (compound) obtained by assembling this tree representation can be uniquely decided to be NH₂CH₂NH₂.

Further, for the singleton, the site information is not required but may be added in implementation.

The restoration unit 108, 210 performs restoration based on the site information.

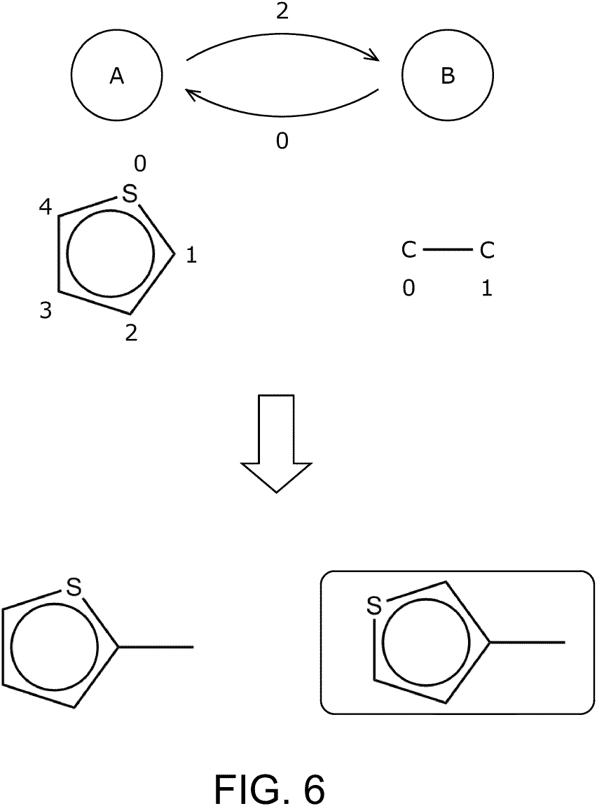

FIG. 6 is a diagram illustrating an example of the site information, and illustrates the site information regarding the ring-bond connection. Node A is a ring and node B is a bond.

“2” of node A is obtained by giving numbers in such a manner to pass through all of the atoms starting from a predetermined atom in the ring of the graph of node A. As illustrated in FIG. 6, numbers 0, 1, ..., 4 are given in order starting from S in the diagram. For node B, numbers 0, 1 are given similarly to the case in FIG. 5.

2 being the number of the atom to be connected is added as the site information to the edge from node A toward node B, and 0 is added as the site information to the edge from node B toward node A.

In the case of having the above site information, the restoration unit 108, 210 can uniquely restore not a left graph but a right graph in a lower diagram from the information on the tree with site information. The restoration unit 108, 210 connects, for example, an atom at position 2 of node A based on the information on the edge from node A toward node B with an atom at position 0 of node B based on the information on the edge from node B toward node A.

In the case of connection of the ring and the bond, the restoration unit 108, 210 adds the position of the atom to be connected in the ring as the site information as above and thereby can uniquely restore the atomic graph from the information on the tree.

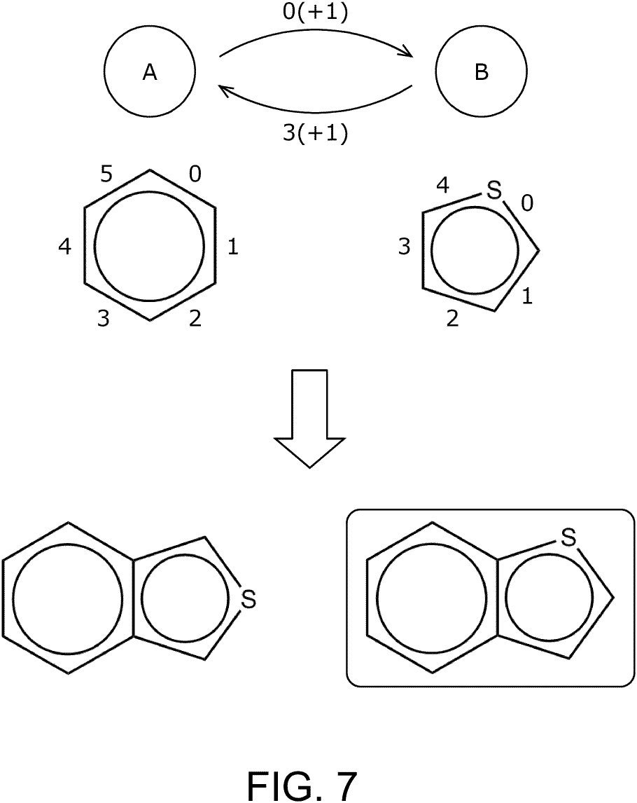

FIG. 7 is a diagram illustrating an example of the site information, and illustrates the site information regarding the ring-ring condensation connection. Both of node A and node B are rings. For example, node A is a 6-membered ring aromatic compound and node B is a 5-membered ring aromatic compound.

In node A, numbers 0 to 5 are added in order from a certain side in the atomic graph. Also in node B, numbers 0 to 4 are similarly added. Unlike FIG. 6, the number as the site information is added not to the node of the atomic graph but to the edge of the atomic graph.

In the case of the condensation connection, the number of the edge in the atomic graph and the direction for connection are designated as the site information of the node. For example, number 0 and direction +1 are added as the site information on edge A → B. Number 3 and direction +1 are similarly added as the site information on edge B → A. The direction means whether the connection is made in the order of the added numbers or in the reverse order. For example, the direction is given clockwise, but as illustrated only, and may be given counterclockwise as long as it can uniquely designate the connection.

It is assumed that in the example in FIG. 7, if the site information on edge A → B is 0(+1) and the site information on edge B → A is 3(+1), 0 in the atomic graph of node A is connected in direction + and 3 in the atomic graph of node B is connected in direction +. Therefore, the restoration unit 108, 210 can uniquely restore a connection state on a lower right side in the diagram.

In the case of the example in FIG. 7, when the site information on edge A → B is 0(+1), the site information on edge B → A may be set to 1(-1) for indicating the same connection. As above, there may be a plurality of ways to give the site information regarding the same connection as long as it is possible to uniquely restore the atomic graph from the information on the tree including the site information.

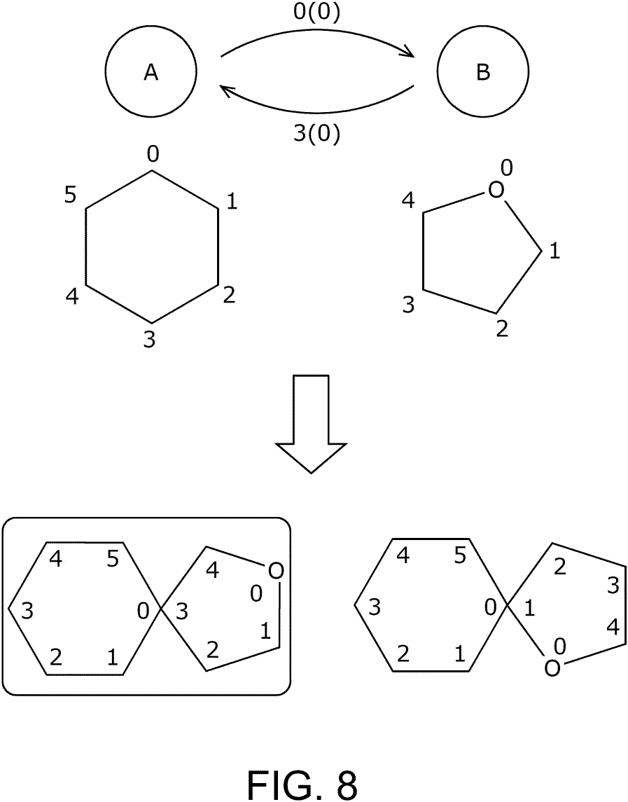

FIG. 8 is a diagram illustrating an example of the site information, and illustrates the site information regarding the ring-ring spiro connection. Both of node A and node B are rings, and have the same configuration as in FIG. 7.

In the case of the spiro connection, the site direction information is set to 0. When restoring the tree indicating the ring-ring to a graph, the restoration unit 108, 210 may first refer to the site direction information. When the site direction information is 0, the restoration unit 108, 210 determines that the site information indicates the number of the atom, and connects the designated atoms to each other to restore the graph information as illustrated in FIG. 8. On the other hand, when the site direction information is ±1, the restoration unit 108, 210 connects the edges to each other based on the case in FIG. 7 to restore the graph information.

### Training Method

By performing the tree decomposition with site information as above, the training method of the neural network model (encoder NN, decoder NN) is also changed from the general machine learning method.

As one example, TreeGRU (Tree Gated Recurrent Unit) is used for training the above information with site and, for example, even Tree LSTM (Tree Long Short Term Memory) can be similarly mounted. The TreeGRU uses, for example, a method described in W. Jin, et.al., “Junction Tree Variational Autoencoder for Molecular Graph Generation,” arXiv: 1802.04364v4, Mar. 29, 2019. For example, in the TreeGRU, optimization of the autoencoder by VAE can be realized. These are illustrated as examples only, and any appropriate network formation methods and optimization methods are applicable.

In this embodiment, the training can be expressed by the following equations.

$${\text{h}}_{i j} = \text{EdgeTreeGRU} \left({\text{x}}_{i} , {\left\{{\text{e}}_{i j}\right\}}_{k ∈ N \left(i\right) / j} , {\left\{{\text{h}}_{i j}\right\}}_{k ∈ N \left(i\right) / j}\right)$$

$${\text{k}}_{i j} = {\sum }_{k ∈ N \left(i\right) \backslash \text{} j} \left[{\text{h}}_{k i} , {\text{e}}_{k i}\right]$$

$${\text{z}}_{i j} = σ \left({\text{W}}^{z} {\text{x}}_{i} + {\text{U}}^{z} {\text{k}}_{i j} + {\text{b}}^{z}\right)$$

$${\text{r}}_{i j} = σ \left({\text{W}}^{r} {\text{x}}_{i} + {\text{U}}^{r} \left[{\text{h}}_{i j} , {\text{e}}_{i j}\right] + {\text{b}}^{r}\right)$$

$${\text{m}}_{i j} = tanh \left({\text{Wx}}_{i} + \text{U} \sum {}_{k ∈ N \left(i\right) / j} {\text{r}}_{k i} ⊙ \left[{\text{h}}_{k i} , {\text{e}}_{k i}\right]\right)$$

$${\text{h}}_{i j} = \left(1 - {\text{z}}_{i j}\right) ⊙ {\text{k}}_{i j} + {\text{z}}_{i j} ⊙ {\text{m}}_{i j}$$

EdgeTreeGRU() in Equation (1) is GRU designed to input/output the tree representation with site information as a message. x is a vector indicating a feature amount indicating the kind or the like of the node, and is expressed, for example, by a one-hot vector. e is information on an edge, namely, a vector indicating a feature amount of the site information (including the site direction information) and is expressed, for example, by a one-hot vector. h is a message vector between nodes. By defining a feature amount vector including the site information and giving the message vector between the nodes as a hidden vector of the GRU as above, the processing is executed similarly to the GRU.

Further, σ() in each equation indicates a sigmoid function, and odot indicates a product of elements. W, U represent weights, and b represents a bias term.

According to Equation (1) to Equation (6), the message vector h is calculated. Here, the site information is calculated while being concatenated so that the message vector of the GRU includes the information as indicated in Equation (2), Equation (4), and Equation (5).

The encoding unit 206 forms the encoder NN 220 using the EdgeTreeGRUO in the above Equation (1) as the network representing the GRU. The decoding unit 208 also similarly forms the decoder NN 222 using the EdgeTreeGRU(). The decoding unit 208 executes decoding of the site information together with the decoding of the graph information based on the following equations.

$${p}_{t} = σ \left({\text{u}}^{d} ⋅ τ \left({\text{W}}_{1}^{d} {\text{x}}_{i t} + {\text{W}}_{2}^{d} {\text{z}}_{T} + {\text{W}}_{3}^{d} \sum {}_{\left(k , {i}_{t}\right) ∈ {ε}_{t}} {\text{h}}_{k , {i}_{t}}\right)\right)$$

$${\text{q}}_{j} = \text{softmax} \left({\text{u}}^{l} ⋅ τ \left({\text{W}}_{1}^{l} {\text{z}}_{T} + {\text{W}}_{2}^{l} {\text{h}}_{i j}\right)\right)$$

$${\tilde{\text{s}}}_{j} = τ \left({\text{W}}_{1}^{s} {\text{z}}_{T} + {\text{W}}_{2}^{l} {\text{h}}_{i j}\right)$$

$${\text{s}}_{j} = \text{softmax} \left({\text{u}}^{s} ⋅ {\tilde{\text{s}}}_{j}\right)$$

$${\text{d}}_{j} = \text{softmax} \left({\text{u}}^{d} ⋅ {\tilde{\text{s}}}_{j}\right)$$

In each equation, u, W represent weights.

In Equation (7), T() represents ReLU. In decoding a certain node, the decoding unit 208 calculates a probability of whether the node further has a child node based on the output z at the preceding step, the feature x of the node, and the received message h, based on Equation (7).

In Equation (8), q represents the feature of the node when the child node is generated.

Further, in this embodiment, the decoding unit 208 infers the site information and the site direction information in addition to the tree node information.

The decoding unit 208 calculates an intermediate variable using the output at the preceding step and the input message based on Equation (9).

The decoding unit 208 further acquires the site information from a result of Equation (9) and the weight based on Equation (10) and infers the site direction information from the result of Equation (9) and the weight based on Equation (11).

As a result of this, the decoding unit 208 acquires the information on the tree with site from the latent variable. The above is the operations of the encoding unit 206 and the decoding unit 208 as the autoencoder, but it is also possible to use only the decoder NN. In the inferring device 1, the decoding unit 106 acquires the information on the tree with site based on the calculations of Equation (7) to Equation (11) for the latent variable.

The update unit 212 calculates an evaluation value (Loss) of the information on the tree encoded and decoded based on the above equations with respect to the training data, and updates the weights U, W, u in the equations and, in some cases, the bias b, based on the evaluation value. The loss is expressed by the following equations.

$${L}_{c} \left(T\right) {\sum }_{t} {L}^{d} \left({p}_{t} , {\hat{p}}_{t}\right) + {\sum }_{j} {L}^{l} \left({q}_{i} , {\hat{q}}_{j}\right)$$

$${L}_{c s} \left(T\right) = {L}_{c} \left(T\right) + {w}_{s} {\sum }_{j} {L}^{s} \left({\text{s}}_{j} , {\hat{\text{s}}}_{j}\right) + {w}_{d} {\sum }_{j} {L}^{k} \left({\text{d}}_{j} , {\hat{\text{d}}}_{j}\right)$$

p_hat, q_hat, s_hat, and d_hat represent grand truth values with respect to predicted values p, q, s, and d, respectively. wₛ and w_{d} are hyperparameters for adjusting the balance between the site information and the site direction information, and need to be appropriately set. Further, each L is a loss function appropriately set for each variable. In Equation (13), a loss function is defined which is obtained by modifying Equation (12) being the loss function about a general TLSTM, in order to use the information with site.

To minimize a cross entropy loss expressed in Equation (13), the update unit 212 updates the parameters of the network. By repeating the operation, the update unit 212 executes optimization of the encoder NN 220 and the decoder NN 222 (decoder NN 120). For example, q_hat can be acquired by the decomposition unit 204 decomposing the atomic graph acquired as the training data into a tree with site.

The optimization by the update unit 212 is executed using a general method. For example, a method of Teacher Forcing of inputting correct answer data into the next step of the GRU may be used. As a matter of course, other methods such as Scheduled Sampling, Professor Forcing can be used.

### Encoding

The site information is explained using FIG. 5 to FIG. 8, and how to encode the site information will be explained next. The training reflecting the tree information with site information in the above training can be executed by using the following encoding. The decomposition unit 204 adds the site information and the site direction information to the nodes becoming the bond, the singleton, and the ring at the timing of the tree decomposition when the atomic graph is input.

### One Direction

The site information may be given in one direction. The decomposition unit 204 encodes the site information on one side on one edge. There are a method of encoding the site information on a departure side of the edge and a method of encoding the site information on an arrival side of the edge.

In the case of encoding the site information on the departure side of the edge, (site of node A, site direction) is given as the edge feature to the edge from node A to node B, whereby the site information is encoded.

For example, the site information given to the edge from node A to node B in FIG. 6 is encoded as (2, 0), and the site information given to the edge from node B to node A is encoded as (0, 0).

For example, the site information given to the edge from node A to node B in FIG. 7 is (0, +1), and the site information given to the edge from node B to node A is (3, +1). Alternatively, the site information given to the edge from node B to node A may be (1, -1).

For example, the site information given to the edge from node A to node B in FIG. 8 is (0, 0), and the site information given to the edge from node B to node A is (3, 0). In this case, the site direction is set to 0 as explained above, thereby indicating the spiro connection, and it is possible to read that the information indicated by the site is not the edge of the graph but the node of the graph.

In the case of encoding the site information on the arrival side of the edge, (site of node B, site direction) is given as the edge feature to the edge from node A to node B, whereby the site information is encoded.

For example, the site information given to the edge from node A to node B in FIG. 6 is encoded as (0, 0), and the site information given to the edge from node B to node A is encoded as (2, 0).

For example, the site information given to the edge from node A to node B in FIG. 7 is (3, +1), and the site information given to the edge from node B to node A is (0, +1).

As explained above, the site information can be given using the information seen from the own node. Further, for example, in the situation in FIG. 6, if the site information on one side is given, the site information on the other side is not required. For example, since the atom to be connected is the same atom, the same atom in the node on the other side may be extracted from the site information on one side, and the result may be regarded as the site information. As explained above, the site information can be omitted according to the situation in some cases.

### Both Directions

Further, not the site information only on the own node side or only on the partner node side as above, but the site information on both sides may be given as the feature of the edge. In other words, the site information on both sides may be encoded on one directed edge.

For example, the site information given to the edge from node A to node B in FIG. 6 is encoded as (2, 0, 0) and the site information given to the edge from node B to node A is encoded as (0, 2, 0).

For example, the site information given to the edge from node A to node B in FIG. 7 is encoded as (0, 3, +1) and the site information given to the edge from node B to node A is encoded as (3, 0, +1).

The site direction may be calculated, for example, by the numbers given to the nodes of the graph. It is assumed that adjacent atomic nodes in node A are ai and aj (i and j are atomic node numbers and i < j, or i is a maximum atomic node number and j = 0). Further, it is assumed that adjacent atomic nodes in node B are bl and bk (l and k are atomic node numbers). On those assumptions, a case where the bond of ai-aj and the bond of bl-bk are connected is considered as one example.

When l < k, the site direction is assumed to be +1. For example, the site information on the edge from node A to node B becomes (i, l, +1). On the other hand, when l > k, the site direction is assumed to be -1. For example, the site information on the edge from node A to node B becomes (i, l, -1). However, when one of l and k is 0 and the other is a maximum atomic node number, vice versa.

The acquisition of the site direction information as above makes it possible to uniquely restore the decomposed information on the tree to the graph. Note that giving the site direction is explained as one example, and the method is not limited to the above method but only needs to be an appropriate giving method capable of uniquely converting the site direction information to the graph at the bond condensed and connected in the ring.

Also in this case, when the site information on one side exists, the site information on the other side is not required in the case of FIG. 6.

It is desirable, but not limited, to use the site information in both directions in the training. This is considered to be because the information on a node connected a certain node is not given by the information from the node on the partner side but can be read from both nodes owing to the site information in both directions in decoding.

In the inferring device 1 or the training device 2, the restoration unit 108, 210 re-configures the atomic graph based on the information on the tree with site output from the decoding unit. For example, the nodes may be restored in sequence starting from the first node of the graph by an autoregressive method. In this embodiment, since the site information exists in addition to the information on the tree, the inference from each node to the next node can be uniquely decided. The inference of the node only needs to be performed by a reverse operation to the above giving the site information.

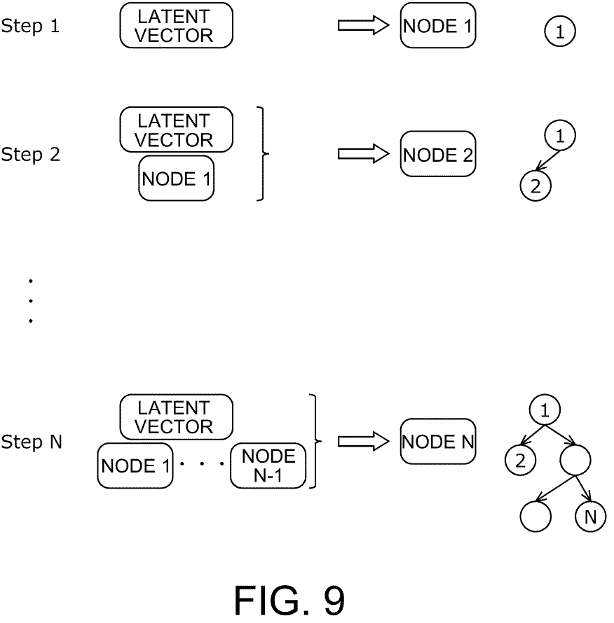

FIG. 9 is a diagram for explaining the re-configuration of the atomic graph. The generation of the tree structure may be executed by the same method as an autoregressive model of RNN (Recurrent Neural Network).

First, the restoration unit generates node 1 by the neural network model of the decoding unit as a predetermined origin node from an acquired latent vector.

Next, the restoration unit autoregressively generates node 2 based on the latent vector and on the information on node 1. At this step, the restoration unit acquires a structure of a tree connecting node 1 and node 2 as illustrated on the right side of the diagram. For the generation of node 2, the neural network model having an autoregressive configuration may be used as represented by the RNN.

The restoration unit repeats this operation until all of nodes (for example, the nodes to node N) are generated. The repeated calculation makes it possible to acquire the tree structure, namely, to acquire a molecular structure.

As explained above, according to this embodiment, giving the site information at the timing of decomposing the atomic graph into a tree makes it possible to uniquely realize the restoration from the information on the tree to the atomic graph. In this embodiment, the method of tree decomposition with site is proposed as the method of solving the unique restoration. Further, the use of the autoencoder as the method of inferring the information on the tree decomposition with site from the latent variable, is explained.

The use of the method in this embodiment makes it possible to speedily restore the information on the atomic graph from the information on the tree. Thus, by learning mapping from the latent representation to the compound, it becomes possible to construct a molecular generative model usable for designing a new compound. The construction of the model can be speedily realized for various compound groups. Further, using the learned model makes it possible to speedily generate and design a new compound. This model is applicable to drug discovery and material search.

The trained models of above embodiments may be, for example, a concept that includes a model that has been trained as described and then distilled by a general method.

Some or all of each device (the inference device 1 or the training device 2) in the above embodiment may be configured in hardware, or information processing of software (program) executed by, for example, a CPU (Central Processing Unit), GPU (Graphics Processing Unit). In the case of the information processing of software, software that enables at least some of the functions of each device in the above embodiments may be stored in a non-volatile storage medium (non-volatile computer readable medium) such as CD-ROM (Compact Disc Read Only Memory) or USB (Universal Serial Bus) memory, and the information processing of software may be executed by loading the software into a computer. In addition, the software may also be downloaded through a communication network. Further, entire or a part of the software may be implemented in a circuit such as an ASIC (Application Specific Integrated Circuit) or FPGA (Field Programmable Gate Array), wherein the information processing of the software may be executed by hardware.

A storage medium to store the software may be a removable storage media such as an optical disk, or a fixed type storage medium such as a hard disk, or a memory. The storage medium may be provided inside the computer (a main storage device or an auxiliary storage device) or outside the computer.

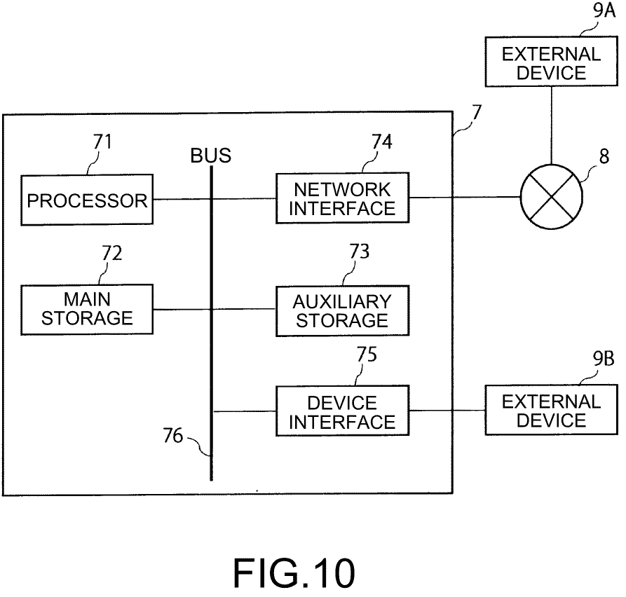

FIG. 10 is a block diagram illustrating an example of a hardware configuration of each device (the inference device 1 or the training device 2) in the above embodiments. As an example, each device may be implemented as a computer 7 provided with a processor 71, a main storage device 72, an auxiliary storage device 73, a network interface 74, and a device interface 75, which are connected via a bus 76.

The computer 7 of FIG. 10 is provided with each component one by one but may be provided with a plurality of the same components. Although one computer 7 is illustrated in FIG. 10, the software may be installed on a plurality of computers, and each of the plurality of computer may execute the same or a different part of the software processing. In this case, it may be in a form of distributed computing where each of the computers communicates with each of the computers through, for example, the network interface 74 to execute the processing. That is, each device (the inference device 1 or the training device 2) in the above embodiments may be configured as a system where one or more computers execute the instructions stored in one or more storages to enable functions. Each device may be configured such that the information transmitted from a terminal is processed by one or more computers provided on a cloud and results of the processing are transmitted to the terminal.

Various arithmetic operations of each device (the inference device 1 or the training device 2) in the above embodiments may be executed in parallel processing using one or more processors or using a plurality of computers over a network. The various arithmetic operations may be allocated to a plurality of arithmetic cores in the processor and executed in parallel processing. Some or all the processes, means, or the like of the present disclosure may be implemented by at least one of the processors or the storage devices provided on a cloud that can communicate with the computer 7 via a network. Thus, each device in the above embodiments may be in a form of parallel computing by one or more computers.

The processor 71 may be an electronic circuit (such as, for example, a processor, processing circuity, processing circuitry, CPU, GPU, FPGA, or ASIC) that executes at least controlling the computer or arithmetic calculations. The processor 71 may also be, for example, a general-purpose processing circuit, a dedicated processing circuit designed to perform specific operations, or a semiconductor device which includes both the general-purpose processing circuit and the dedicated processing circuit. Further, the processor 71 may also include, for example, an optical circuit or an arithmetic function based on quantum computing.

The processor 71 may execute an arithmetic processing based on data and / or a software input from, for example, each device of the internal configuration of the computer 7, and may output an arithmetic result and a control signal, for example, to each device. The processor 71 may control each component of the computer 7 by executing, for example, an OS (Operating System), or an application of the computer 7.

Each device (the inference device 1 or the training device 2) in the above embodiments may be enabled by one or more processors 71. The processor 71 may refer to one or more electronic circuits located on one chip, or one or more electronic circuitries arranged on two or more chips or devices. In the case of a plurality of electronic circuitries are used, each electronic circuit may communicate by wired or wireless.

The main storage device 72 may store, for example, instructions to be executed by the processor 71 or various data, and the information stored in the main storage device 72 may be read out by the processor 71. The auxiliary storage device 73 is a storage device other than the main storage device 72. These storage devices shall mean any electronic component capable of storing electronic information and may be a semiconductor memory. The semiconductor memory may be either a volatile or non-volatile memory. The storage device for storing various data or the like in each device (the inference device 1 or the training device 2) in the above embodiments may be enabled by the main storage device 72 or the auxiliary storage device 73 or may be implemented by a built-in memory built into the processor 71. For example, the storages 102, 202 in the above embodiments may be implemented in the main storage device 72 or the auxiliary storage device 73.

In the case of each device (the inference device 1 or the training device 2) in the above embodiments is configured by at least one storage device (memory) and at least one of a plurality of processors connected/coupled to/with this at least one storage device, at least one of the plurality of processors may be connected to a single storage device. Or at least one of the plurality of storages may be connected to a single processor. Or each device may include a configuration where at least one of the plurality of processors is connected to at least one of the plurality of storage devices. Further, this configuration may be implemented by a storage device and a processor included in a plurality of computers. Moreover, each device may include a configuration where a storage device is integrated with a processor (for example, a cache memory including an L1 cache or an L2 cache).

The network interface 74 is an interface for connecting to a communication network 8 by wireless or wired. The network interface 74 may be an appropriate interface such as an interface compatible with existing communication standards. With the network interface 74, information may be exchanged with an external device 9A connected via the communication network 8. Note that the communication network 8 may be, for example, configured as WAN (Wide Area Network), LAN (Local Area Network), or PAN (Personal Area Network), or a combination of thereof, and may be such that information can be exchanged between the computer 7 and the external device 9A. The internet is an example of WAN, IEEE802.11 or Ethernet (registered trademark) is an example of LAN, and Bluetooth (registered trademark) or NFC (Near Field Communication) is an example of PAN.

The device interface 75 is an interface such as, for example, a USB that directly connects to the external device 9B.

The external device 9A is a device connected to the computer 7 via a network. The external device 9B is a device directly connected to the computer 7.

The external device 9A or the external device 9B may be, as an example, an input device. The input device is, for example, a device such as a camera, a microphone, a motion capture, at least one of various sensors, a keyboard, a mouse, or a touch panel, and gives the acquired information to the computer 7. Further, it may be a device including an input unit such as a personal computer, a tablet terminal, or a smartphone, which may have an input unit, a memory, and a processor.

The external device 9A or the external device 9B may be, as an example, an output device. The output device may be, for example, a display device such as, for example, an LCD (Liquid Crystal Display), or an organic EL (Electro Luminescence) panel, or a speaker which outputs audio. Moreover, it may be a device including an output unit such as, for example, a personal computer, a tablet terminal, or a smartphone, which may have an output unit, a memory, and a processor.

Further, the external device 9A or the external device 9B may be a storage device (memory). The external device 9A may be, for example, a network storage device, and the external device 9B may be, for example, an HDD storage.

Furthermore, the external device 9A or the external device 9B may be a device that has at least one function of the configuration element of each device (the inference device 1 or the training device 2) in the above embodiments. That is, the computer 7 may transmit a part of or all of processing results to the external device 9A or the external device 9B, or receive a part of or all of processing results from the external device 9A or the external device 9B.

In the present specification (including the claims), the representation (including similar expressions) of “at least one of a, b, and c” or “at least one of a, b, or c” includes any combinations of a, b, c, a - b, a - c, b - c, and a - b - c. It also covers combinations with multiple instances of any element such as, for example, a - a, a - b -b, or a - a - b - b - c - c. It further covers, for example, adding another element d beyond a, b, and / or c, such that a - b - c - d.

In the present specification (including the claims), the expressions such as, for example, “data as input,” “using data,” “based on data,” “according to data,” or “in accordance with data” (including similar expressions) are used, unless otherwise specified, this includes cases where data itself is used, or the cases where data is processed in some ways (for example, noise added data, normalized data, feature quantities extracted from the data, or intermediate representation of the data) are used. When it is stated that some results can be obtained “by inputting data,” “by using data,” “based on data,” “according to data,” “in accordance with data” (including similar expressions), unless otherwise specified, this may include cases where the result is obtained based only on the data, and may also include cases where the result is obtained by being affected factors, conditions, and / or states, or the like by other data than the data. When it is stated that “output/outputting data” (including similar expressions), unless otherwise specified, this also includes cases where the data itself is used as output, or the cases where the data is processed in some ways (for example, the data added noise, the data normalized, feature quantity extracted from the data, or intermediate representation of the data) is used as the output.

In the present specification (including the claims), when the terms such as “connected (connection)” and “coupled (coupling)” are used, they are intended as non-limiting terms that include any of “direct connection / coupling,” “indirect connection / coupling,” “electrically connection / coupling,” “communicatively connection / coupling,” “operatively connection / coupling,” “physically connection / coupling,” or the like. The terms should be interpreted accordingly, depending on the context in which they are used, but any forms of connection / coupling that are not intentionally or naturally excluded should be construed as included in the terms and interpreted in a non-exclusive manner.

In the present specification (including the claims), when the expression such as “A configured to B,” this may include that a physically structure of A has a configuration that can execute operation B, as well as a permanent or a temporary setting / configuration of element A is configured / set to actually execute operation B. For example, when the element A is a general-purpose processor, the processor may have a hardware configuration capable of executing the operation B and may be configured to actually execute the operation B by setting the permanent or the temporary program (instructions). Moreover, when the element A is a dedicated processor, a dedicated arithmetic circuit, or the like, a circuit structure of the processor or the like may be implemented to actually execute the operation B, irrespective of whether or not control instructions and data are actually attached thereto.

In the present specification (including the claims), when a term referring to inclusion or possession (for example, “comprising / including,” “having,” or the like) is used, it is intended as an open-ended term, including the case of inclusion or possession an object other than the object indicated by the object of the term. If the object of these terms implying inclusion or possession is an expression that does not specify a quantity or suggests a singular number (an expression with a or an article), the expression should be construed as not being limited to a specific number.

In the present specification (including the claims), although when the expression such as “one or more,” “at least one,” or the like is used in some places, and the expression that does not specify a quantity or suggests a singular number (the expression with a or an article) is used elsewhere, it is not intended that this expression means “one.” In general, the expression that does not specify a quantity or suggests a singular number (the expression with a or an as article) should be interpreted as not necessarily limited to a specific number.

In the present specification, when it is stated that a particular configuration of an example results in a particular effect (advantage / result), unless there are some other reasons, it should be understood that the effect is also obtained for one or more other embodiments having the configuration. However, it should be understood that the presence or absence of such an effect generally depends on various factors, conditions, and / or states, etc., and that such an effect is not always achieved by the configuration. The effect is merely achieved by the configuration in the embodiments when various factors, conditions, and / or states, etc., are met, but the effect is not always obtained in the claimed invention that defines the configuration or a similar configuration.

In the present specification (including the claims), when the term such as “maximize / maximization” is used, this includes finding a global maximum value, finding an approximate value of the global maximum value, finding a local maximum value, and finding an approximate value of the local maximum value, should be interpreted as appropriate accordingly depending on the context in which the term is used. It also includes finding on the approximated value of these maximum values probabilistically or heuristically. Similarly, when the term such as “minimize” is used, this includes finding a global minimum value, finding an approximated value of the global minimum value, finding a local minimum value, and finding an approximated value of the local minimum value, and should be interpreted as appropriate accordingly depending on the context in which the term is used. It also includes finding the approximated value of these minimum values probabilistically or heuristically. Similarly, when the term such as “optimize” is used, this includes finding a global optimum value, finding an approximated value of the global optimum value, finding a local optimum value, and finding an approximated value of the local optimum value, and should be interpreted as appropriate accordingly depending on the context in which the term is used. It also includes finding the approximated value of these optimal values probabilistically or heuristically.

In the present specification (including claims), when a plurality of hardware performs a predetermined process, the respective hardware may cooperate to perform the predetermined process, or some hardware may perform all the predetermined process. Further, a part of the hardware may perform a part of the predetermined process, and the other hardware may perform the rest of the predetermined process. In the present specification (including claims), when an expression (including similar expressions) such as “one or more hardware perform a first process and the one or more hardware perform a second process,” or the like, is used, the hardware that perform the first process and the hardware that perform the second process may be the same hardware, or may be the different hardware. That is: the hardware that perform the first process and the hardware that perform the second process may be included in the one or more hardware. Note that, the hardware may include an electronic circuit, a device including the electronic circuit, or the like.

In the present specification (including the claims), when a plurality of storage devices (memories) store data, an individual storage device among the plurality of storage devices may store only a part of the data or may store the entire data. Further, some storage devices among the plurality of storage devices may include a configuration for storing data.

While certain embodiments of the present disclosure have been described in detail above, the present disclosure is not limited to the individual embodiments described above. Various additions, changes, substitutions, partial deletions, etc. are possible to the extent that they do not deviate from the conceptual idea and purpose of the present disclosure derived from the contents specified in the claims and their equivalents. For example, when numerical values or mathematical formulas are used in the description in the above-described embodiments, they are shown for illustrative purposes only and do not limit the scope of the present disclosure. Further, the order of each operation shown in the embodiments is also an example, and does not limit the scope of the present disclosure.

1. An inferring device comprising:
one or more memories; and
one or more processors configured to:
generate information on a tree including information on a node and information on an edge from a latent representation by using a trained inference model; and
generate a graph from the information on the tree, wherein
the information on the tree includes connection information on the nodes.

2. The inferring device according to claim 1, wherein the graph is a graph of a molecular structure.

3. The inferring device according to claim 2, wherein:
the node is any one of
a singleton node representing an atom indicating a branch point in the graph of the molecular structure,
a bond node representing a node other than the singleton of acyclic atomic nodes, and
a ring node representing a cyclic atom structure; and the connection of the nodes is any one of
the singleton node and the bond node,
the bond node and the bond node,
the bond node and the ring node, and
the ring node and the ring node.

4. The inferring device according to claim 1, wherein:
the connection information on the nodes includes a direction of connecting bond in a case where ring nodes are connected by sharing a bond belonging to both the ring nodes.

5. The inferring device according to claim 1, wherein the one or more processors
generate the latent representation from a second latent representation including information on a second tree including information on a node and information on an edge.

6. The inferring device according to claim 1, wherein
the one or more processors generate the latent representation by using random values.

7. The inferring device according to claim 1, wherein
the one or more processors generate a plurality of pieces of information on a tree from the plurality of latent representations in parallel.

8. The inferring device according to claim 1, wherein
the connection information on the nodes includes information on a connection position of the nodes connected by the edge and information on a connection direction of the nodes.

9. The inferring device according to claim 1, wherein
the latent representation includes a latent variable.

10. The inferring device according to claim 1, wherein
the trained inference model is a neural network having an autoregressive configuration.

11. The inferring device according to claim 10, wherein
the one or more processors generate the information on the tree autoregressively using the neural network.

12. The inferring device according to claim 11, wherein
the one or more processors input the latent representation and information on generated nodes into the neural network.

13. A training device comprising:
one or more memories; and
one or more processors configured to:
generate information on a first tree including information on a first node and information on a first edge from a graph;
generate a latent representation based on a first network from the information on the first tree;
generate information on a second tree including information on a second node and information on a second edge based on a second network from the latent representation; and
update parameters of the first network and the second network based on a result of comparison between input information into the first network and output information from the second network.

14. The training device according to claim 13, wherein
the information on the first edge includes connection information on the first nodes connected by the first edge.

15. The training device according to claim 14, wherein
the connection information on the first nodes includes information on a connection position of the first nodes connected by the first edge and information on a connection direction of the first nodes.

16. The training device according to claim 13, wherein
the second neural network is a neural network having an autoregressive configuration.

17. The training device according to claim 14, wherein:
the connection information on the first nodes includes a direction of connecting bond in a case where ring nodes are connected by sharing a bond belonging to both the ring nodes.

18. An inferring method comprising:
generating, by one or more processors, information on a tree including information on a node and information on an edge from a latent representation by using a trained inference model; and
generating, by the one or more processors, a graph from the information on the tree,
wherein the information on the tree includes connection information on the nodes.

19. The inferring method according to claim 18, wherein
the connection information on the nodes includes a direction of connecting bond in a case where ring nodes are connected by sharing a bond belonging to both the ring nodes.

20. The inferring method according to claim 18, wherein
the connection information on the nodes includes information on a connection position of the nodes connected by the edge and information on a connection direction of the nodes.
